(19) United States
(12) Patent Application Publication
(10) Pub. No.: US 2020/0297446 A1
(43) Pub. Date: Sep. 24, 2020

(54) Method and Apparatus for Providing Improved Peri-operative Scans and Recall of Scan Data

(71) Applicant: TransEnterix Surgical, Inc., Morrisville, NC (US)

(72) Inventors: Damien Brassett, Milano (IT); Stefano Rivera, Milano (IT); Kevin Andrew Hufford, Cary, NC (US)

(21) Appl. No.: 16/018,042

(22) Filed: Jun. 25, 2018

Related U.S. Application Data
(60) Provisional application No. 62/524,154, filed on Jun. 23, 2017.

(51) Int. Cl. A61B 34/00; A61B 34/37; A61B 34/10; A61B 1/04; A61B 1/00; A61B 1/06

(52) U.S. Cl. CPC A61B 34/74; A61B 34/37; A61B 34/10; A61B 2034/301; A61B 1/04; A61B 1/00045; A61B 1/06; A61B 34/76

(57) ABSTRACT

In a system and method of displaying peri-operative and real-time data, capturing peri-operative 3D scan data of a region of interest in a body cavity is captured using a scanning instrument. A 3D scan map is generated using the 3D scan data. During a surgical procedure real-time images of a body cavity are captured using an endoscopic camera mounted to a robotic arm and are displayed on a display. The 3D scan map is selectively displayed as an overlay on the displayed real-time images

### BACKGROUND

Various surface mapping methods exist that allow the topography of a surface to be determined. One type of surface mapping method is one using structured light. Structured light techniques are used in a variety of contexts to generate three-dimensional (3D) maps or models of surfaces. These techniques include projecting a pattern of structured light (e.g. a grid or a series of stripes) onto an object or surface. One or more cameras capture an image of the projected pattern. From the captured images the system can determine the distance between the camera and the surface at various points, allowing the topography/shape of the surface to be determined.

In surgery, it is useful to provide an overall scan of the patient abdomen near the beginning of a procedure, or before beginning a step in the procedure. Recall of this model may then be used for a variety of purposes. For example, data from such a scan may be used to create a “world model” for a surgical robotic system and be used to identify structures in the abdomen that are to be avoided by robotically controlled instruments. Such a system, which may include configurations that anticipate the possibility of instrument contact with such structures as well as those that cause the robotic system to automatically avoid the structures, is described in co-pending U.S. application Ser. No. 16/010,388, filed Jun. 15, 2018, which is incorporated herein by reference.

The methods described herein may be used with surgical robotic systems. There are different types of robotic systems on the market or under development. Some surgical robotic systems use a plurality of robotic arms. Each arm carries a surgical instrument, or the camera used to capture images from within the body for display on a monitor. Other surgical robotic systems use a single arm that carries a plurality of instruments and a camera that extend into the body via a single incision. Each of these types of robotic systems uses motors to position and/or orient the camera and instruments and to, where applicable, actuate the instruments. Typical configurations allow two or three instruments and the camera to be supported and manipulated by the system. As with manual laparoscopic surgery, surgical instruments and cameras used for robotic procedures may be passed into the body cavity via trocars. Input to the system is generated based on input from a surgeon positioned at a master console, typically using input devices such as input handles and a foot pedal. Motion and actuation of the surgical instruments and the camera is controlled based on the user input. The system may be configured to deliver haptic feedback to the surgeon at the controls, such as by causing the surgeon to feel resistance at the input handles that is proportional to the forces experienced by the instruments moved within the body. The image captured by the camera is shown on a display at the surgeon console. The console may be located patient-side, within the sterile field, or outside of the sterile field.

### BRIEF DESCRIPTION OF THE DRAWINGS

FIG. 1 schematically illustrates the flow of information in a system using principles of the invention.

FIG. 2 schematically illustrates a method for scanning and acquiring data using multiple viewpoints.

FIG. 3 is an example of an endoscopic image.

FIG. 4 shows the image of FIG. 3 which is obscured because of significant blood in the surgical site.

FIG. 5 shows the image of FIG. 4, in which an overlay is placed over the endoscopic image.

### DETAILED DESCRIPTION

This application describes the use of surface mapping techniques to aid the surgeon in planning and/or performing a procedure. For example, the described system and methods can give an overall scan of the patient abdomen near the beginning of a procedure, or before beginning a step in the procedure. Recall of this model may then be used by the surgeon for a variety of purposes. The described methods may be performed using a robotic surgical system, although they can also be implemented without the use of surgical robotic systems.

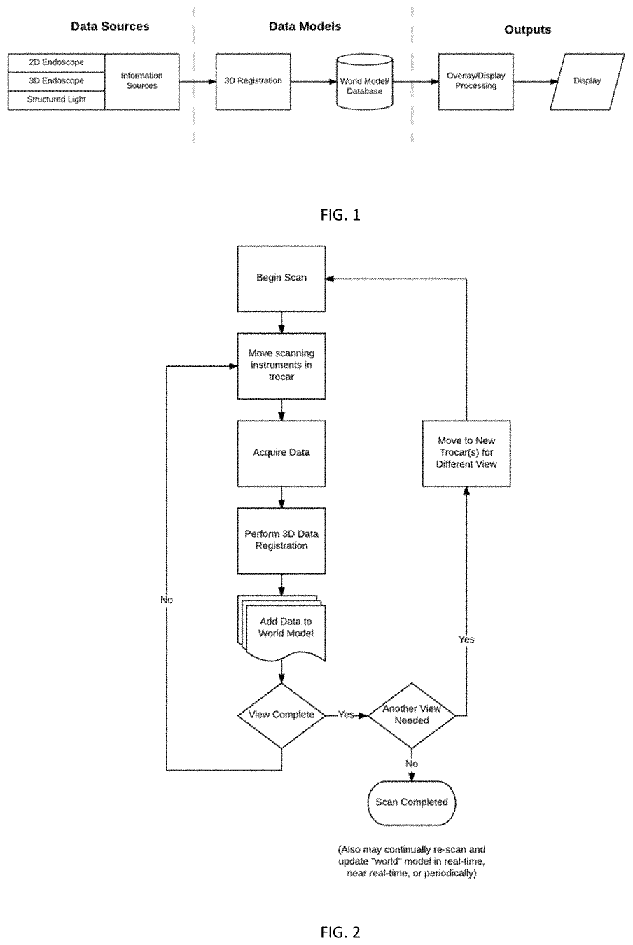

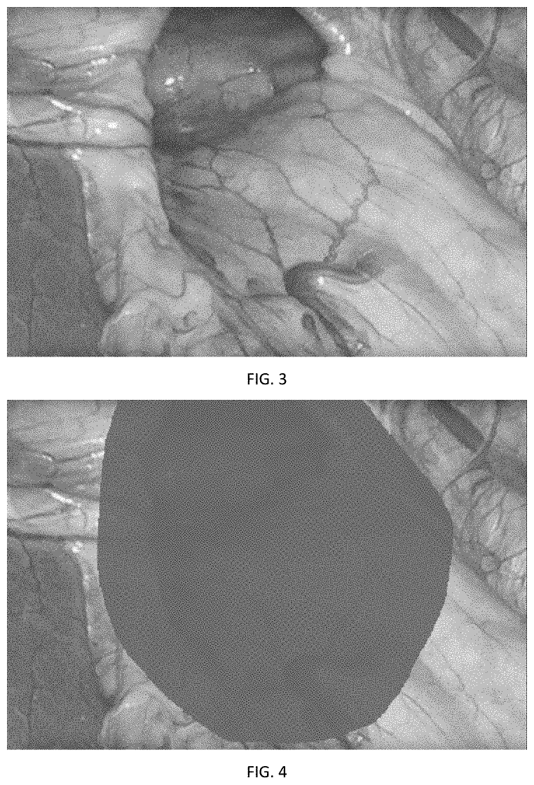

FIG. 1 illustrates the flow of information in a system using principles of the invention. Data sources include image data sources, surface position/shape/topography data, and other information sources. Data may be obtained by a video-based acquisition, either 2D or 3D, or may include other technologies such as structured light, for improved accuracy.

In some cases, multiple scans of the cavity are collected to allow collection of images/data from multiple vantage points. In such cases, data from a plurality of scans is co-registered into an overall 3-dimensional model of the surgical site. The 3-dimensional model may form part of a world model of the surgical site and used for a variety of purposes. For example, the user might choose to have the system recall a prior scan and display it as an overlay to the real-time endoscope view shown on the system's endoscopic display. The user interface might include input features (e.g. a fingerwheel/thumbwheel or knob on the user interface, a touch screen interface, eye tracking control, etc) allowing the user to select a desired level of opacity of the overlay. Alternatively, the opacity might be altered based on some sensed parameter(s) or condition(s) within the imaging field, such as the change in shape of a structure or area in the surgical site.

In laparoscopic surgery and in robotic surgical applications, a number of trocars are positioned within small incisions formed in the abdomen. Each instrument receives a camera or surgical instrument used to perform a procedure within the abdomen. In some implementations of the concepts disclosure here, multiple trocars may be employed during the process of generating the scan.

FIG. 2 schematically illustrates a method for scanning and acquiring data using multiple viewpoints. The source(s) of the data for the scan (each referenced generally here as a “scanning instrument”) may be an endoscopic camera, which may be a 2D endoscopic view or a 3D endoscopic camera. Additionally, the addition of a structured light source may be used to add higher fidelity data and improved depth information beyond just a 2D overlay. Structured light techniques result in data sets representing the shape/topography of the tissue and the position of tissue surfaces.

To enhance the usability of a visualization means, it may be helpful to use multiple vantage points in acquiring imagery and/or data. In some embodiments, multiple trocars are used to allow scans to be captured from different vantage points. For example, an endoscope may be moved between a series of trocars, capturing scans from each trocar position, to improve the points visible in an overall scan. In some implementations, a source of structured light may be inserted into one or more trocars. In other embodiments, sources of structured light may be disposed in multiple trocars and alternately illuminated. In some implementations, an endoscope and a source of structured light may be moved from one trocar to another trocar (or multiple moves may be made to multiple trocars) to improve the points visible in the overall scan. Trocars configured to project structured light onto tissue surfaces are described in U.S. application Ser. No. 16/010,388, entitled Method and Apparatus for Trocar-Based Structured Light Applications, incorporated herein by reference, may also be used.

During or after the scanning procedure or sequence, feedback may be given to the user about the suitability of a scan/the comprehensiveness of a scan. On-screen prompts may provide overlays about the coverage (e.g. the area to be captured by the scan based on current scanning instrument positions), provide cueing inputs for a scan, and/or walk the user through a series of steps (e.g. instructing the user to move the scanning instruments between trocars).

In some implementations, the robotic surgical system may perform an autonomous move or series of moves to move cameras and, as applicable, structure light source(s). With this configuration, the system can scan around a wide view, a smaller region, or a particular region of interest. The user may be prompted to select from a menu of such scan types prior to initiating the scan. The scanning process or sequence may be pre-programmed and/or capable of being modified by the user. The scan is preferably done during the surgical procedure, and may be performed before or after the surgeon begins to work on tissue within the body cavity.

Data obtained through the scanning procedure may be used for a number of reasons, including, but not limited to, training purposes and simulation. This data may also be used to measure the size and/or positions of structures in the surgical site, and to identify structures that should be avoided during the procedure as described above.

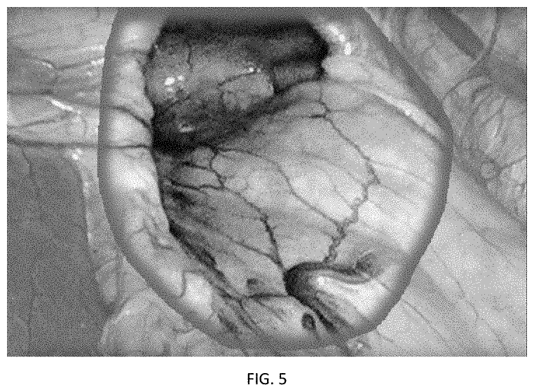

Another use is to provide the user with a temporary view of a region that might be obstructed in the real-time endoscope view at a given point in time. For example, if the surgical view becomes obscured as in FIG. 4, such as by the surgical site filling with blood, the scanned view may be recalled as an overlay to the endoscopic view at a controllable opacity as shown in FIG. 5. This may also be useful to enhance contrast or visibility when the abdomen is filled with surgical smoke from the use of electrosurgical equipment. The overlay may be over the entire endoscopic image, a portion of the image determined to be occluded, or some selected region of interest.

Co-pending U.S. application Ser. No. 16/010,388 filed Jun. 15, 2018, which is incorporated herein by reference, describes creation, and use of a “world model”, or a spatial layout of the environment within the body cavity, which includes the relevant anatomy and tissues/structures within the body cavity that are to be avoided by surgical instruments during a robotic surgery. The systems and methods described in this application may provide 3D data for the world model or associated kinematic models in that (see for example FIG. 5 of that application) type of system and process. See, also, FIG. 2 herein, in which the world view is updated based on the acquired data.

The described system/method may also incorporate the automatic or assisted detection of occlusions or other anatomical structures/features from “Method of Graphically Tagging and Recalling Identified Structures Under Visualization for Robotic Surgery,” U.S. Ser. No. 16/______, (TRX-16010), filed Jun. 25, 2018, which is incorporated herein by reference.

1-6. (canceled)

7. A surgical system comprising:
an endoscopic camera mounted to a robotic arm; and
an endoscopic camera display system configured to display real-time images captured using the endoscopic camera, and to selectively display a peri-operative 3D surface scan map generated using data captured by a scanning instrument, the scan map displayed as an overlay to the real-time images.

8. The system of claim 7, wherein the overlay has an opacity, and wherein the system includes a user input device operable to input a user selection of an opacity level, the system responsive to input from the user input device to set the opacity level of the overlay.

9. The system of claim 8, wherein the user input device is a fingerwheel, thumbwheel, knob or other input element on a manually-operated user handle used to give input on movement of surgical instruments within an operative site.

10. The system of claim 7, wherein the overlay has an opacity, and wherein the system is configured to alter the level of opacity based on a detection of a condition in the endoscopic view.

11. The system of claim 7, wherein the overlay has an opacity, and wherein the system is configured to alter the level of opacity based on detection by the system of a change in shape of a structure or area in the body cavity.

12. The system of claim 7, wherein the scanning instrument comprises a 3-dimensional endoscope.

13. The system of claim 7, wherein the scanning instrument comprises a 2-dimensional endoscope and a source of structured light.

14. The system of 7, wherein in which the scanning instrument comprises a 3-dimensional endoscope and a source of structured light.

15-16. (canceled)

17. The system of claim 7, wherein the surface scan map is generating through a scanning sequence performed pre-operatively by capturing scans of the abdominal cavity from multiple vantage points.

18. The system of claim 17, wherein the scanning sequence includes robotically moving the scanning instrument within the body cavity to capture scans from the multiple vantage points.

19-23. (canceled)

24. A method of displaying peri-operative and real-time data comprising:
capturing peri-operative 3D scan data of a region of interest in a body cavity using a scanning instrument;
generating a 3D scan map using the 3D scan data;
during a surgical procedure, capturing real-time images of a body cavity using an endoscopic camera mounted to a robotic arm; and
displaying the real-time images on a display;
selectively displaying the 3D scan map as an overlay on the displayed real-time images.

25. The method of claim 24, wherein the method includes receiving input from a user corresponding to selection of an opacity level, and displaying the overlay with an opacity corresponding to the selected opacity level.

26. The method of claim 24, wherein the overlay has an opacity, and wherein the method includes altering a degree of the opacity based on a detection by the system of a change in shape of a structure or area in the endoscopic view.

27. The method of claim 24, wherein capturing the scan data and generating the scan map include performing a scanning sequence by capturing scans of the abdominal cavity from multiple vantage points.

28. The method of claim 27, wherein the scanning sequence includes robotically moving the scanning instrument within the body cavity to capture scans from the multiple vantage points.

29. The method of claim 28, wherein the scanning sequence includes moving the scanning instrument(s) from a first trocar to a second trocar and capturing scans from a position in each trocar.

30. The method of claim 29, further including moving the scanning instrument from the second trocar to a third trocar and capturing a scan from the third trocar.

31. The method of claim 29, wherein scan data captured from the first and trocar position(s) at least partially overlaps.

32. The method of claim 29, wherein scan data captured from the first and trocar position(s) is non-overlapping.

33. The method of claim 24 wherein the data from scans captured in the scanning sequence are co-registered into an overall 3-dimensional model of the surgical site.
